(19) United States
(12) Patent Application Publication
(10) Pub. No.: US 2026/0306553 A1
(43) Pub. Date: Oct. 8, 2026

(54) Chinese medicine composition for dissolving and excreting urate crystals in the body and preparation method thereof

(71) Applicant: Yuhua Xie, Chenzhou (CN)

(72) Inventor: Yuhua Xie, Chenzhou (CN)

(21) Appl. No.: 19/098,252

(22) Filed: Apr. 2, 2025

(51) Int. Cl. A61K 35/583; A61K 33/00; A61K 35/14; A61K 36/076; A61K 36/28; A61K 36/31; A61K 36/744; A61K 36/8967; A61K 36/8994

(52) U.S. Cl. CPC A61K 35/583; A61K 33/00; A61K 35/14; A61K 36/076; A61K 36/28; A61K 36/31; A61K 36/744; A61K 36/8967; A61K 36/8994; A61K 2236/15; A61K 2236/331; A61K 2236/51

(57) ABSTRACT

A Chinese medicine composition for dissolving and excreting urate crystals in the body, a preparation method, and an application thereof; wherein the drugs comprise 1 part to 30 parts of deer blood, 1 part to 30 parts of zaocys dhumnade, 1 part to 30 parts of semen raphani, 1 part to 30 parts of poria cocos, 1 part to 30 parts of coix seed, 1 part to 30 parts of chicory, 1 part to 30 parts of lily, 1 part to 30 parts of gardenia, and 1 part to 30 parts of baking soda. The invention has a reasonable combination, fully utilizes the pharmacological effects of various drugs, synergistically enhances efficacy, achieves dissolution and excretion of urate crystals in the body, alleviates gout symptoms, reduces uric acid levels, and improves the overall health status of patients.

### 1. TECHNICAL FIELD

The invention relate to the technical field of Chinese medicine composition, and in particular to a Chinese medicine composition for dissolving and excreting urate crystals in the body and a preparation method thereof.

### 2. BACKGROUND ART

As the number of people with hyperuricemia continues to increase, the disease has caused long-term damage to people's health and brought a heavy social burden. By forming urate crystals in the body, hyperuricemia causes a series of complications such as pain, gouty arthritis, joint deformity, urinary calculus, arteriosclerosis, vascular obstruction, etc. Therefore, developing a method that configured to dissolve and excrete urate crystals in the body is of great significance for truly achieving the purpose of treating gout and reversing the complications of hyperuricemia.

### 3. SUMMARY OF THE INVENTION

The purpose of the invention is to provide a safe formula for dissolving and excreting urate crystals in the body and a preparation method thereof, effectively addressing hyperuricemia and its complications, and filling the gaps in existing technology. To solve the technical problems mentioned hereinabove, the invention provides the following technical solution:

A Chinese medicine composition for dissolving and excreting urate crystals in the body, comprising the following components in parts by weight:
1 part to 30 parts of deer blood, 1 part to 30 parts of zaocys dhumnade, 1 part to 30 parts of semen raphani, 1 part to 30 parts of poria cocos, 1 part to 30 parts of coix seed, 1 part to 30 parts of chicory, 1 part to 30 parts of lily, 1 part to 30 parts of gardenia, and 1 part to 30 parts of baking soda.

As a preferred embodiment of the invention, the Chinese medicine composition also comprises pharmaceutically acceptable excipients.

As a preferred embodiment of the invention, the Chinese medicine composition is prepared into capsules, tablets, granules or decoctions.

A preparation method of the invention, comprising the following steps:
S1: crushing
crushing zaocys dhumnade, semen raphani, chicory, and gardenia to obtain medicinal powder;
S2: decoting
decocting deer blood, poria cocos, coix seed, lily, and baking soda with water, and filtering and concentrating the filtrate into a clear paste;
S3: drying
vacuum drying the powder and the clear paste, then crushing, and mixing the obtained materials in proportion to finally obtain the finished product.

As a preferred embodiment of the invention, in the decocting step, the amount of water added in S2 is 5 times to 10 times the total weight of the Chinese medicine, and the decoction time is 1 hour to 3 hours.

As a preferred embodiment of the invention, in the drying step, the vacuum drying temperature in S3 is 50° C. to 80° C., and the drying time is 3 minutes to 6 minutes.

In the drugs of the invention:
deer blood: it is sweet and salty in taste, warm in nature, which acts on liver and kidney meridians; it has the effects of nourishing blood and boosting essence, stopping bleeding, and reducing the secretion of leucorrhea; it configured to treat symptoms such as insufficient essence and blood, lumbago, impotence and seminal emission, palpitation due to deficiency of blood, insomnia, etc;
zaocys dhumnade: it is sweet in taste, gentle in nature, which acts on liver meridian; it has the effects of eliminating pathogenic wind, dredging meridian, controlling or relieving muscle spasms; it configured to treat stubborn rheumatism, spasms accompanied by numbness symptoms, facial paralysis caused by stroke, convulsive spasm;
semen raphani: it has the effects of digesting food and eliminating bloating, lowing lung qi and resolving phlegm; it configured to treat symptoms such as food stagnation, abdominal distension, constipation, cough and asthma;
poria cocos: it is sweet and light in taste, gentle in nature, which act on the heart, lungs, spleen, and kidney meridians; it has the effects of promoting diuresis and eliminating dampness, strengthening the spleen and calming the mind; it configured to treat edema, dysuria, spleen deficiency diarrhea, palpitations and insomnia, etc;
coix seed: it is sweet and light in taste, cool in nature, which acts on the spleen, stomach, lungs, large intestine, and small intestine meridians; it has the effects of promoting diuresis and eliminating dampness, strengthening the spleen and removing rheumatism, clearing away heat and eliminating pus; it configured to treat edema, beriberi, dysuria, spasms caused by dampness, etc;
chicory: it has the effects of eliminating damp heat in the liver and gallbladder, strengthening the stomach and digestion, and promoting diuresis and reducing swelling; it configured to treat damp-heat in the liver and gallbladder, loss of appetite, edema and oliguria, etc;
lily: it is sweet in taste, cold in nature, which acts on the heart and lung meridians; it has the effects of nourishing lung yin and relieving lung dryness, easing mental anxiety; it configured to treat cough caused by lung yin deficiency, cough due to fatigue and hemoptysis, restlessness and palpitations, insomnia and dreaminess, etc;
gardenia: it is bitter in taste, cold in nature, which acts on the heart, lung, and triple energizer meridians; it has the effects of eliminating accumulated heat in the body and relieving restlessness, eliminating dampness and heat from the body, clearing heat and detoxifying; it configured to treat restlessness caused by fever, dark yellow urine color caused by jaundice, and pain in the urethra during urination;
baking soda: it also known as sodium bicarbonate, which has the effects of neutralizing gastric acid and alkalizing urine; it configured to treat hyperacidity, metabolic acidosis, etc.

In the invention, deer blood is mainly used to nourish blood and boost essence, and zaocys dhumnade is mainly used to eliminate pathogenic wind, dredging meridian. The combination of the two configured to not only nourish blood and boost essence, but also dispel pathogenic wind, targeting the main symptoms of deficiency and pathogenic wind. Semen raphani configured to digest food and eliminate bloating, low lung qi and resolve phlegm; poria cocos configured to promote diuresis and eliminate dampness, strengthen the spleen and calm the mind; coix seed configured to promote diuresis and eliminate dampness, strengthen the spleen and remove rheumatism; chicory configured to eliminate damp heat in the liver and gallbladder, strengthen the stomach and digestion, and promote diuresis and reduce swelling; these drugs work together to assist deer blood and zaocys dhumnade, enhancing their nourishing and dispelling effects; lily configured to nourish lung yin and relieve lung dryness, ease mental anxiety; gardenia configured to eliminate accumulated heat in the body and relieving restlessness, eliminate dampness and heat from the body, clear heat and detoxify, which is mainly used to harmonize the properties of other drugs in the prescription, reduce the dryness of the drugs, and regulate possible symptoms such as yin deficiency, cough caused by lung yin deficiency, and restlessness; baking soda is mainly used to harmonize the acidic components in other drugs.

In summary, the invention has a reasonable combination, fully utilizes the pharmacological effects of various drugs, synergistically enhances efficacy, achieves dissolution and excretion of urate crystals in the body, alleviates gout symptoms, reduces uric acid levels, and improves the overall health status of patients. The invention configured to dissolve urate crystals in the body and excrete them outside the body, which configured to reverse a series of complications caused by hyperuricemia such as gouty arthritis, joint deformity, urinary stones, arteriosclerosis, etc., achieving the effect of treating and preventing gout and its complications. It is safe, effective, and configured to be reused for a long time.

### 4. SPECIFIC EMBODIMENT OF THE INVENTION

In order to make the objectives, technical solutions, and advantages of the embodiments of the invention clearer, the technical solutions in the embodiments of the invention will be described clearly and completely hereinafter with reference to the drawings in the embodiments of the invention. Obviously, the described embodiments are part of the embodiments of the invention, rather than all of the embodiments. The components of the embodiments of the invention generally described and illustrated in the drawings herein may be arranged and designed in various different configurations.

### Embodiment 1

This embodiment provides a Chinese medicine composition for dissolving and excreting urate crystals in the body, wherein the components are prepared according to the following weight proportions:
15 grams of deer blood, 15 grams of zaocys dhumnade, 15 grams of semen raphani, 15 grams of poria cocos, 15 grams of coix seed, 15 grams of chicory, 15 grams of lily, 15 grams of gardenia, and 15 grams of baking soda.

The preparation method of this embodiment is as follows:
S1: crushing
crushing zaocys dhumnade, semen raphani, chicory, and gardenia to obtain medicinal powder;
S2: Decocting
decocting deer blood, poria cocos, coix seed, lily, and baking soda with water, and filtering and concentrating the filtrate into a clear paste; in this step, the amount of water added is 5 times to 10 times the total weight of the Chinese medicine, and the decoction time is 1 hour to 3 hours.
S3: drying
vacuum drying the powder and the clear paste, the vacuum drying temperature in S3 is 50° C. to 80° C., and the drying time is 3 minutes to 6 minutes; then crushing, mixing the obtained materials in proportion, and filling into capsules to make capsules.

### Embodiment 2

This embodiment provides a Chinese medicine composition for dissolving and excreting urate crystals in the body, wherein the components are prepared according to the following weight proportions:
10 grams of deer blood, 10 grams of zaocys dhumnade, 10 grams of semen raphani, 10 grams of poria cocos, 10 grams of coix seed, 10 grams of chicory, 30 grams of lily, 30 grams of gardenia, and 30 grams of baking soda.

The preparation method of this embodiment is the same as steps S1 to S2 in embodiment 1, except that after drying in S3, drug carriers such as corn starch are mixed in and then pressed into tablets.

### Embodiment 3

This embodiment provides a Chinese medicine composition for dissolving and excreting urate crystals in the body, wherein the components are prepared according to the following weight proportions:
30 grams of deer blood, 30 grams of zaocys dhumnade, 30 grams of semen raphani, 30 grams of poria cocos, 30 grams of coix seed, 30 grams of chicory, 10 grams of lily, 10 grams of gardenia, and 10 grams of baking soda.

The preparation method of this embodiment is the same as steps S1 to S2 in embodiment 1, except that after drying in S 3, it is prepared into granules or soup.

The embodiments thereof described hereinabove are only preferred embodiments of the invention and are not intended to limit it. Any modifications, equivalent substitutions, and improvements made within the spirit and principles of the invention patent should be included within the scope of protection of the invention.

1. A Chinese medicine composition for dissolving and excreting urate crystals in the body, comprising the following components in parts by weight:

1. part to 30 parts of deer blood, 1 part to 30 parts of zaocys dhumnade, 1 part to 30 parts of semen raphani, 1 part to 30 parts of poria cocos, 1 part to 30 parts of coix seed, 1 part to 30 parts of chicory, 1 part to 30 parts of lily, 1 part to 30 parts of gardenia, and 1 part to 30 parts of baking soda.

2. The Chinese medicine composition of claim 1, wherein the Chinese medicine composition also comprises pharmaceutically acceptable excipients.

3. The Chinese medicine composition of claim 1, wherein the Chinese medicine composition is prepared into capsules, tablets, granules or decoctions.

4. A preparation method of the Chinese medicine composition of claim 1, comprising the following steps:
S1: crushing
crushing zaocys dhumnade, semen raphani, chicory, and gardenia to obtain medicinal powder;
S2: decocting
decocting deer blood, poria cocos, coix seed, lily, and baking soda with water, and filtering and concentrating the filtrate into a clear paste;
S3: drying
vacuum drying the powder and the clear paste, then crushing, and mixing the obtained materials in proportion to finally obtain the finished product.

5. The Chinese medicine composition of claim 4, wherein in the decocting step, the amount of water added in S2 is 5 times to 10 times the total weight of the Chinese medicine, and the decoction time is 1 hour to 3 hours.

6. The Chinese medicine composition of claim 4, wherein in the drying step, the vacuum drying temperature in S3 is 50° C. to 80° C., and the drying time is 3 minutes to 6 minutes.

7. An application of the Chinese medicine composition of claim 1 in the preparation of a medicine for treating hyperuricemia and its complications.

8. The application of the Chinese medicine composition of claim 7, wherein the complications of hyperuricemia comprise gouty arthritis, joint deformity, urinary calculus, arteriosclerosis, and vascular obstruction.

9. The application of the Chinese medicine composition of claim 7, wherein the medicine is in the form of capsules, tablets, granules or decoctions.
